(19) United States
(12) Patent Application Publication
(10) Pub. No.: US 2017/0221204 A1
(43) Pub. Date: Aug. 3, 2017

(54) Overlay Of Findings On Image Data

(71) Applicant: Siemens Medical Solutions USA, Inc., Malvern, PA (US)

(72) Inventor: Yoshihisa Shinagawa, Downingtown, PA (US)

(21) Appl. No.: 15/413,486

(22) Filed: Jan. 24, 2017

Related U.S. Application Data
(60) Provisional application No. 62/287,917, filed on Jan. 28, 2016.

(51) Int. Cl. G06T 7/00; G06K 9/62; A61B 6/00; G06F 17/27; A61B 5/00; G06T 11/60; G06K 9/00

(52) U.S. Cl. CPC G06T 7/0012; G06T 11/60; G06K 9/6218; G06K 9/00463; G06F 17/2775; G06F 17/2705; G06F 17/2785; A61B 5/7267; A61B 6/5217; A61B 6/5229; G06T 2207/10004; G06T 2207/30176; G06K 2209/05

(57) ABSTRACT

A framework for overlaying findings on image data is described herein. In accordance with one aspect, the framework extracts one or more findings from a radiology report, and detects one or more anatomical landmarks in image data corresponding to the radiology report. The one or more extracted findings are then correlated to, and overlaid with, the one or more detected anatomical landmarks on the image data.

### CROSS-REFERENCE TO RELATED APPLICATION

The present application claims the benefit of U.S. provisional application No. 62/287,917 filed Jan. 28, 2016, the entire contents of which are herein incorporated by reference.

### TECHNICAL FIELD

The present disclosure generally relates to digital medical image data processing, and more particularly to overlay of findings on image data.

### BACKGROUND

The field of medical imaging has seen significant advances since the time X-Rays were first used to determine anatomical abnormalities. Medical imaging hardware has progressed from modern machines, such as Magnetic Resonance (MR) imaging scanners, Computed Tomographic (CT) scanners and Positron Emission Tomographic (PET) scanners, to multimodality imaging systems such as PET-CT and PET-Mill systems. Because of large amount of image data generated by such modern medical scanners, there has been and remains a need for developing image processing techniques that can automate some or all of the processes to determine the presence of anatomical abnormalities in scanned medical images.

Digital medical images are constructed using raw image data obtained from a scanner, for example, a computerized axial tomography (CAT) scanner, magnetic resonance imaging (MRI), etc. Digital medical images are typically either a two-dimensional (“2D”) image made of pixel elements, a three-dimensional (“3D”) image made of volume elements (“voxels”) or a four-dimensional (“4D”) image made of dynamic elements (“doxels”). Such 2D, 3D or 4D images are processed using medical image recognition techniques to determine the presence of anatomical abnormalities or pathologies, such as cysts, tumors, polyps, etc. Given the amount of image data generated by any given image scan, it is preferable that an automatic technique should point out anatomical features in the selected regions of an image to a doctor for further diagnosis of any disease or condition.

Automatic image processing and recognition of structures within a medical image is generally referred to as Computer-Aided Detection (CAD). A CAD system can process medical images, localize and segment anatomical structures, including possible abnormalities (or candidates), for further review. Recognizing anatomical structures within digitized medical images presents multiple challenges. For example, a first concern relates to the accuracy of recognition of anatomical structures within an image. A second area of concern is the speed of recognition. Because medical images are an aid for a doctor to diagnose a disease or condition, the speed with which an image can be processed and structures within that image recognized can be of the utmost importance to the doctor in order to reach an early diagnosis.

When a radiologist opens a new case associated with a patient, typical tasks include reading the radiology reports from previous examinations of the same patient, loading associated images to a workstation, and visiting locations of previously reported abnormalities or pathologies. These tasks are tedious and time-consuming, particularly because the radiologist typically has to almost memorize the findings reported in the previous examinations before reviewing the images.

### SUMMARY

Described herein is a framework for overlaying findings on image data. In accordance with one aspect, the framework extracts one or more findings from a radiology report, and detects one or more anatomical landmarks in image data corresponding to the radiology report. The one or more extracted findings are then correlated to, and overlaid with, the one or more detected anatomical landmarks on the image data.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 is a block diagram illustrating an exemplary system;

FIG. 2 shows an exemplary method of overlaying findings on image data by a computer system; and

FIG. 3 shows an exemplary user interface screen with overlaid images.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed; on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance.

The term “x-ray image” as used herein may mean a visible x-ray image (e.g., displayed on a video screen) or a digital representation of an x-ray image (e.g., a file corresponding to the pixel output of an x-ray detector). The term “in-treatment x-ray image” as used herein may refer to images captured at any point in time during a treatment delivery phase of an interventional or therapeutic procedure, which may include times when the radiation source is either on or off. From time to time, for convenience of description, CT imaging data (e.g., cone-beam CT imaging data) may be used herein as an exemplary imaging modality. It will be appreciated, however, that data from any type of imaging modality including but not limited to x-ray radiographs, MRI, PET (positron emission tomography), PET-CT, SPECT, SPECT-CT, MR-PET, 3D ultrasound images or the like may also be used in various implementations.

Unless stated otherwise as apparent from the following discussion, it will be appreciated that terms such as “segmenting,” “generating,” “registering,” “determining,” “aligning,” “positioning,” “processing,” “computing,” “selecting,” “estimating,” “detecting,” “tracking” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present framework are not described with reference to any particular programming language. It will be appreciated that a variety of programming languages may be used.

As used herein, the term “image” refers to multi-dimensional data composed of discrete image elements (e.g., pixels for 2D images and voxels for 3D images). The image may be, for example, a medical image of a subject collected by computer tomography, magnetic resonance imaging, ultrasound, or any other medical imaging system known to one of skill in the art. The image may also be provided from non-medical contexts, such as, for example, remote sensing systems, electron microscopy, etc. Although an image can be thought of as a function from R³ to R, or a mapping to R³, the present methods are not limited to such images, and can be applied to images of any dimension, e.g., a 2D picture or a 3D volume. For a 2- or 3-dimensional image, the domain of the image is typically a 2- or 3-dimensional rectangular array, wherein each pixel or voxel can be addressed with reference to a set of 2 or 3 mutually orthogonal axes. The terms “digital” and “digitized” as used herein will refer to images or volumes, as appropriate, in a digital or digitized format acquired via a digital acquisition system or via conversion from an analog image.

The terms “pixels” for picture elements, conventionally used with respect to 2D imaging and image display, and “voxels” for volume image elements, often used with respect to 3D imaging, can be used interchangeably. It should be noted that the 3D volume image is itself synthesized from image data obtained as pixels on a 2D sensor array and displayed as a 2D image from some angle of view. Thus, 2D image processing and image analysis techniques can be applied to the 3D volume image data. In the description that follows, techniques described as operating upon pixels may alternately be described as operating upon the 3D voxel data that is stored and represented in the form of 2D pixel data for display. In the same way, techniques that operate upon voxel data can also be described as operating upon pixels. In the following description, the variable x is used to indicate a subject image element at a particular spatial location or, alternately considered, a subject pixel. The terms “subject pixel” or “subject voxel” are used to indicate a particular image element as it is operated upon using techniques described herein.

A framework for automatically overlying findings on image data is described herein. In accordance with one aspect, the framework overlays findings described in radiology reports on corresponding medical image data. The findings may be correlated to and positioned at or near anatomical landmarks detected in the image data. Advantageously, the radiologist (or other user) does not have to memorize the findings from the radiology reports, and can instead concentrate on examining the images. These and other features and advantages will be described in more details herein.

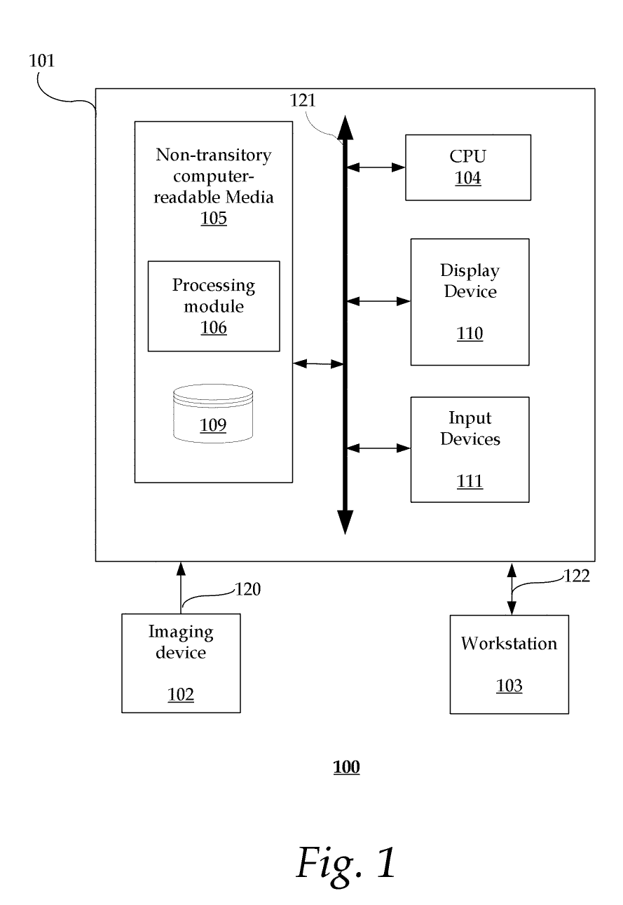

FIG. 1 is a block diagram illustrating an exemplary system 100. The system 100 includes a computer system 101 for implementing the framework as described herein. In some implementations, computer system 101 operates as a standalone device. In other implementations, computer system 101 may be connected (e.g., using a network) to other machines, such as imaging device 102 and workstation 103. In a networked deployment, computer system 101 may operate in the capacity of a server (e.g., thin-client server), a cloud computing platform, a client user machine in server-client user network environment, or as a peer machine in a peer-to-peer (or distributed) network environment.

In some implementations, computer system 101 comprises a processor or central processing unit (CPU) 104 coupled to one or more non-transitory computer-readable media 105 (e.g., computer storage or memory), display device 110 (e.g., monitor) and various input devices 111 (e.g., mouse or keyboard) via an input-output interface 121. Computer system 101 may further include support circuits such as a cache, a power supply, clock circuits and a communications bus. Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in non-transitory computer-readable media 105. In particular, the present techniques may be implemented by a processing module 106 and a database 109.

Non-transitory computer-readable media 105 may include random access memory (RAM), read-only memory (ROM), magnetic floppy disk, flash memory, and other types of memories, or a combination thereof. The computer-readable program code is executed by CPU 104 to process medical data retrieved from, for example, imaging device 102. As such, the computer system 101 is a general-purpose computer system that becomes a specific purpose computer system when executing the computer-readable program code. The computer-readable program code is not intended to be limited to any particular programming language and implementation thereof. It will be appreciated that a variety of programming languages and coding thereof may be used to implement the teachings of the disclosure contained herein.

The same or different computer-readable media 105 may be used for storing a database (or dataset) 109. Such data may also be stored in external storage or other memories. The external storage may be implemented using a database management system (DBMS) managed by the CPU 104 and residing on a memory, such as a hard disk, RAM, or removable media. The external storage may be implemented on one or more additional computer systems. For example, the external storage may include a data warehouse system residing on a separate computer system, a cloud platform or system, a picture archiving and communication system (PACS), or any other hospital, medical institution, medical office, testing facility, pharmacy or other medical patient record storage system.

Imaging device 102 acquires medical image data 120 associated with at least one patient. Such medical image data 120 may be processed and stored in database 109. Imaging device 102 may be a radiology scanner (e.g., X-ray, MR or a CT scanner) and/or appropriate peripherals (e.g., keyboard and display device) for acquiring, collecting and/or storing such medical image data 120.

The workstation 103 may include a computer and appropriate peripherals, such as a keyboard and display device, and can be operated in conjunction with the entire system 100. For example, the workstation 103 may communicate directly or indirectly with the imaging device 102 so that the medical image data acquired by the imaging device 102 can be rendered at the workstation 103 and viewed on a display device. The workstation 103 may also provide other types of medical data 122 of a given patient. The workstation 103 may include a graphical user interface to receive user input via an input device (e.g., keyboard, mouse, touch screen voice or video recognition interface, etc.) to input medical data 122.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

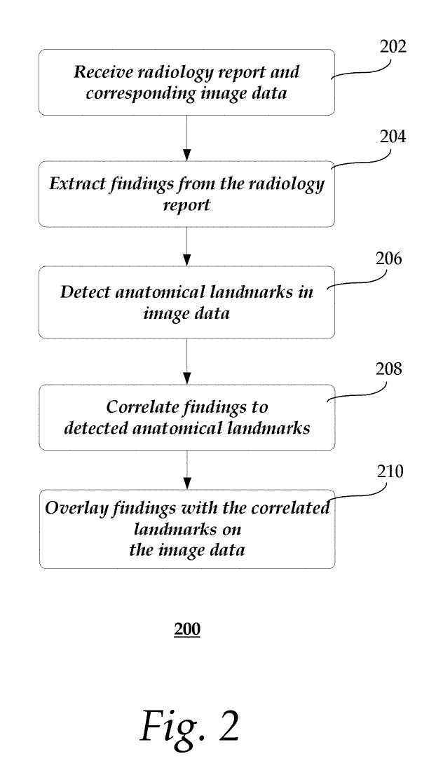

FIG. 2 shows an exemplary method 200 of overlaying findings on image data by a computer system. It should be understood that the steps of the method 200 may be performed in the order shown or a different order. Additional, different, or fewer steps may also be provided. Further, the method 200 may be implemented with the system 101 of FIG. 1, a different system, or a combination thereof.

At 202, processing module 106 receives a radiology report and corresponding image data. The radiology report may be generated by a radiologist who interprets (or reads) the image data. The image data may be acquired during prior examinations of the patient by, for example, imaging device 202 using techniques such as magnetic resonance (MR) imaging, computed tomography (CT), helical CT, X-ray, angiography, positron emission tomography (PET), fluoroscopy, ultrasound, single photon emission computed tomography (SPECT), or a combination thereof.

The radiology report may record various types of clinical information associated with the image data, such as type of examination, clinical history of patient, comparison with previous imaging studies, imaging technique (e.g., whether contrast agent was used), findings, impression (e.g., diagnosis, recommendation), etc. The findings section of the radiology report may list the radiologist's observations regarding each anatomical region examined in the imaging study. The radiologist may include anatomical, disease and/or pathological information that indicates whether each anatomical region was found to be normal, abnormal (or pathological) or potentially abnormal. The impression section of the radiology report typically contains a summary of the findings, and may be processed by processing module 106 similarly to the findings section. The radiology report may be loaded to, for example, workstation 103 to be read by the clinician who ordered the imaging study (or any other user).

At 204, processing module 106 extracts findings from the radiology report. In some implementations, the findings are extracted using a Natural Language Processing (NLP) technique to analyze the text in the radiology report. NLP is a branch of artificial intelligence concerned with analyzing, understanding and generating languages that humans use naturally in order to interface with computers using natural human languages instead of computer languages. Exemplary NLP techniques include, but are not limited to, tagging medical terms, parsing sentences to understand the sentence structures, and analyzing meaning of sentences using machine learning algorithms such as decision trees, statistical models, and so forth. Some of these NLP steps may not be necessary for structured radiology reports where the description is already itemized.

Findings may be extracted by first chunking the text (e.g., findings section, impression section) in the radiology report into sentences, and then parsing the sentences to find anatomical, disease and/or pathological terms that match terms in predefined anatomy, disease and pathology dictionaries. An anatomical term may describe the name of an anatomical region of interest. A disease term may describe one or more abnormalities of the anatomical region. A pathological term may describe a single abnormality often requiring microscopic analysis, while a disease term may refer to a set of multiple pathologies. The anatomical, disease and pathological terms may be found in close proximity in the same phrase (or sub-portion of a sentence) obtained as a result of the sentence parsing. The anatomical, disease and pathological terms may be further modified or augmented by more detailed information, such as etiology, morphology, severity, location, symptoms, description modifiers, and/or treatment. Etiology may describe the triggering events that started the disease.

The extracted findings may be represented as one or more tuples, where each tuple is an ordered list of elements including, but not limited to, anatomical, disease and/or pathological terms, possibly together with modifiers. An exemplary tuple may include elements that describe: {anatomical region, disease or pathology, morphology, severity, etiology, location, description modifier}.

For example, the radiology report may include sentences of findings, such as “there is moderate bilateral pneumothorax and mild pleural effusion, more pronounced on the right. There is a round soft tissue mass within the right thorax.” The findings may be encoded by five tuples: {thorax, pneumothorax, N/A, moderate, N/A, right, more pronounced}, {thorax, pneumothorax, N/A, moderate, N/A, left, N/A}, {thorax, effusion, N/A, mild, N/A, right, more pronounced}, {thorax, effusion, N/A, mild, N/A, left, N/A}, and {thorax, mass, round, N/A, N/A, right, soft tissue}. The term ‘N/A’ indicates that the element is not available in the findings.

Such tuples may be assigned weights according to importance or severity of the findings (or disease). For instance, a finding of probable metastasis is weighted more heavily than a somewhat enlarged organ within normal limits. The weights may be calculated by using machine learning techniques, such as deep learning and support vector machines, and/or based on specified rules. The threshold of weights of the tuples may be adjusted by the users via, for example, a user interface presented at workstation 103. In addition, negations of existence of diseases may also be detected. They may be determined by, for example, detecting predefined keywords (e.g., “no” and “normal”) near or next to an anatomical, disease or pathology term. A negation of existence of diseases may also be detected by NLP and/or machine learning techniques, such as Conditional Random Fields (CRF) or deep learning. For example, sentences such as “there is no pleural effusion,” “the liver is normal” and “the size of the spleen is within normal limits” are determined as negations of existence of disease. Such sentences or terms may be assigned zero (or minimum) weights and/or excluded from the tuples, so that they are not overlaid on the images. The colors, fonts, markers and the brightness of the overlaid text may be changed according to the weights of the findings.

At 206, processing module 106 detects anatomical landmarks in the image data. A landmark is an anatomically meaningful point in the image data. Exemplary anatomical landmarks include, but are not limited to, “left lung apex”, “right lung apex”, “left lung base”, and “right lung base”. To detect the anatomical landmarks, machine learning algorithms (e.g., neural networks, random forests) or other types of algorithms may be applied.

At 208, processing module 106 correlates the extracted findings to the detected anatomical landmarks. As discussed previously, the extracted findings may be represented as one or more tuples. Each tuple may be correlated to an anatomical landmark. For example, the tuple {thorax, pneumothorax, N/A, moderate, N/A, right, more pronounced} may be correlated to the “left lung apex” landmark, and {thorax, effusion, N/A, mild, N/A, left, N/A} may be correlated to “left lung base” landmark. If the detectors of anomalies such as pneumothorax or effusion are available, the tuples may be correlated to the detected locations in the images. In some implementations, one or more predefined rules are employed to associate “thorax” to “lung”. Alternatively, algorithms such as word clustering techniques based on vector representations of words may be used to partition sets of words into clusters (or subsets) of semantically similar words. Tuple terms and landmark terms that are grouped into the same word cluster (e.g., “thorax” and “lung” often belong to the same word cluster) are then correlated.

At 210, processing module 106 overlays the findings and correlated landmarks on the image data. Each landmark may be represented by, for example, a marker, an outline or segmentation of the anatomical region or a text label. Processing module 106 positions findings at or near the corresponding correlated anatomical landmarks on the image data. The choice of findings to overlay may be based on the weights of the tuples determined according to the importance or severity of findings. For example, processing module 106 may select only those tuples with weights that are above a predetermined threshold value to overlay on the image data.

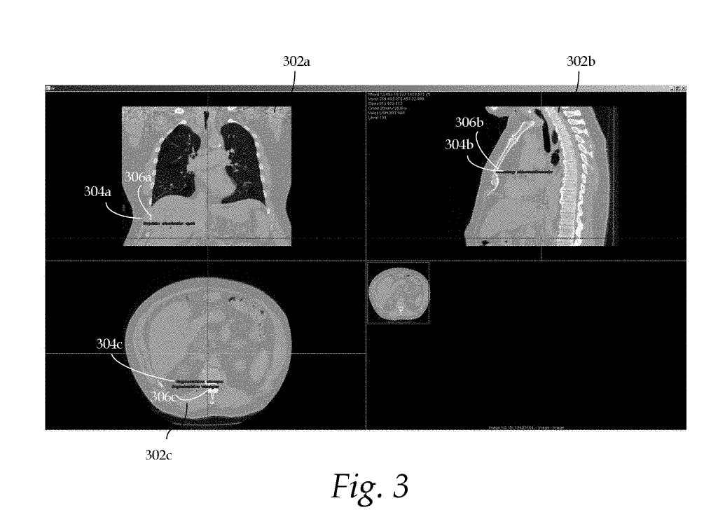

FIG. 3 shows an exemplary user interface screen with overlaid images 302a-c. More particularly, images 302a-c show coronal, sagittal and axial views respectively of a patient's chest. The “hepatic steatosis” tuple (304a) is overlaid on the image 302a near the landmark 306a of the “liver dome” with some displacement. The “degenerative changes” tuple (304b) is overlaid on the image 302b at the “thoracic spine” landmark 306b, while the “coronary atherosclerosis” tuple 304c is overlaid with the “coronary artery” landmark 306c. A radiologist or other user may scroll through the images via, for example, a user interface at workstation 103, to understand the findings from the previous examinations. Alternatively, the radiologist or other user may use the user interface to directly jump to the landmarks so as to expedite the process.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. One or more non-transitory computer readable media embodying a program of instructions executable by machine to perform operations for processing image data, the operations comprising:
extracting one or more findings from a radiology report by performing a Natural Language Processing technique, wherein the one or more extracted findings are represented as one or more tuples;
detecting one or more anatomical landmarks in image data corresponding to the radiology report;
correlating the one or more tuples to the one or more anatomical landmarks; and
overlaying the one or more extracted findings with the correlated one or more anatomical landmarks on the image data.

2. The one or more non-transitory computer readable media of claim 1, wherein performing the Natural Language Processing technique comprises performing machine learning.

3. The one or more non-transitory computer readable media of claim 1, wherein extracting the one or more findings from the radiology report comprises:
chunking text in the radiology report into one or more sentences, and
finding anatomical, disease and pathological terms and their modifiers in the one or more sentences.

4. The one or more non-transitory computer readable media of claim 1, wherein at least one of the one or more tuples comprises an ordered list of elements including an anatomical region of interest, a disease or pathology, a morphology, a severity, an etiology, a location, a description modifier, or a combination thereof.

5. A system comprising:
a non-transitory memory device for storing computer readable program code; and
a processor in communication with the memory device, the processor being operative with the computer readable program code to perform operations including
receiving a radiology report and corresponding image data,
extracting one or more findings from the radiology report,
detecting one or more anatomical landmarks in the image data,
correlating the one or more extracted findings to the one or more anatomical landmarks, and
overlaying the one or more extracted findings with the correlated one or more anatomical landmarks on the image data.

6. The system of claim 5 wherein the findings comprise anatomical, disease and pathological terms associated with one or more anatomical regions.

7. The system of claim 5 wherein the processor is operative with the computer readable program code to extract the one or more findings by performing a Natural Language Processing technique to analyze text in the radiology report.

8. The system of claim 7 wherein the processor is operative with the computer readable program code to perform the Natural Language Processing technique by performing machine learning.

9. The system of claim 5 wherein the processor is operative with the computer readable program code to extract the one or more findings by
chunking text in the radiology report into one or more sentences, and
finding anatomical, disease and pathological terms in the one or more sentences.

10. The system of claim 9 wherein the processor is operative with the computer readable program code to find the anatomical, disease and pathological terms by matching the anatomical, disease and pathological terms with terms in predefined anatomy, disease and pathology dictionaries.

11. The system of claim 5 wherein the processor is operative with the computer readable program code to represent at least one of the one or more extracted findings as a tuple.

12. The system of claim 11 wherein the tuple comprises an ordered list of an anatomical term and one or more disease and pathological terms.

13. The system of claim 12 wherein the one or more disease and pathological terms comprise a disease or pathology, a morphology, a severity, an etiology, a location, a description modifier, or a combination thereof.

14. The system of claim 5 wherein the processor is operative with the computer readable program code to further assign one or more weights to the one or more extracted findings in accordance with importance or severity.

15. The system of claim 14 wherein the processor is operative with the computer readable program code to assign zero weight to at least one of the one or more extracted findings in response to detecting a negation of existence of disease in the extracted finding.

16. The system of claim 14 wherein the processor is operative with the computer readable program code to overlay the one or more extracted findings that are weighted above a predetermined threshold with the correlated one or more anatomical landmarks on the image data.

17. The system of claim 5 wherein the processor is operative with the computer readable program code to correlate the one or more extracted findings to the one or more anatomical landmarks using one or more predefined rules.

18. The system of claim 5, wherein the processor is operative with the computer readable program code to correlate the one or more extracted findings to the one or more anatomical landmarks by
performing a word clustering technique to partition sets of words into clusters of semantically similar words, and
correlating a first term in the one or more extracted findings with a second term describing the one or more anatomical landmarks in response to the first and second terms being grouped into a same word cluster.

19. A method, comprising:
receiving a radiology report and corresponding image data;
extracting one or more findings from the radiology report;
detecting one or more anatomical landmarks in the image data;
correlating the one or more extracted findings to the one or more anatomical landmarks; and
overlaying the one or more extracted findings with the correlated one or more anatomical landmarks on the image data.

20. The method of claim 19 wherein extracting the one or more findings from the radiology report comprises performing a Natural Language Processing technique.
